(19) United States
(12) Patent Application Publication
(10) Pub. No.: US 2026/0165838 A1
(43) Pub. Date: Jun. 18, 2026

(54) DELIVERY DEVICE FOR A REPLACEMENT HEART VALVE IMPLANT

(71) Applicant: BOSTON SCIENTIFIC SCIMED, INC., MAPLE GROVE, MN (US)

(72) Inventors: Levi Joel Wolterstorff, Saint Paul, MN (US); Adam Troy Olson, Plymouth, MN (US); Philip Andrew Litecky, Forest Lake, MN (US); Declan Loughnane, Galway (IE)

(73) Assignee: BOSTON SCIENTIFIC SCIMED, INC., MAPLE GROVE, MN (US)

(21) Appl. No.: 19/421,703

(22) Filed: Dec. 16, 2025

Related U.S. Application Data
(60) Provisional application No. 63/735,568, filed on Dec. 18, 2024.

(51) Int. Cl. A61F 2/24

(52) U.S. Cl. CPC A61F 2/2436

(57) ABSTRACT

A delivery device for a replacement heart valve implant includes a handle assembly and an elongate shaft assembly extending from the handle assembly. The handle assembly includes an actuation mechanism, a distal shell portion, and a friction element engaged with the actuation mechanism and the distal shell portion. First and second amounts of torque are required to rotate the actuation mechanism relative to the distal shell portion in first and second rotational directions. The delivery device may include a plurality of friction elements engaged with the actuation mechanism and the distal shell portion. The friction element may be engaged in compression between the actuation mechanism and the distal shell portion. The friction element is engaged in first and second amounts of compression when the actuation mechanism is rotated in the first and second directions.

### CROSS-REFERENCE TO RELATED APPLICATIONS

This application claims the benefit of priority of U.S. Provisional Application No. 63/735,568 filed Dec. 18, 2024, the entire disclosure of which is hereby incorporated by reference.

### TECHNICAL FIELD

The disclosure relates generally to medical devices and more particularly to medical devices that are adapted for implanting stents and medical devices including a stent component, such as a replacement heart valve implant.

### BACKGROUND

A wide variety of intracorporeal medical devices and/or implants have been developed for medical use including artificial heart valve implants for repair or replacement of diseased heart valves. Some delivery devices may require a number of steps in a particular order to properly deploy an implant. Some users may perform a low volume of cases, and familiarity with the delivery device may be limited. Confusion, cognitive load, and/or stress may slow and/or complicate the procedure. Of the known medical devices, systems, and methods, each has certain advantages and disadvantages. There is an ongoing need to provide alternative devices, systems, and methods for loading medical devices and/or heart valve implants into a delivery system.

### SUMMARY

In one example, a delivery device for delivering a replacement heart valve implant to a native heart valve may comprise a handle assembly and an elongate shaft assembly extending distally from the handle assembly. The handle assembly may comprise a first rotatable knob configured to rotate around a central longitudinal axis of the handle assembly, an actuation mechanism operatively engaged with the first rotatable knob, a distal shell portion disposed radially outward of the actuation mechanism, and a friction element engaged with the actuation mechanism and the distal shell portion. The first rotatable knob may be configured to rotate the actuation mechanism relative to the distal shell portion to axially translate a first portion of the elongate shaft assembly along the central longitudinal axis. A first amount of torque applied to the first rotatable knob may be required to rotate the actuation mechanism relative to the distal shell portion in a first rotational direction, and a second amount of torque applied to the first rotatable knob greater than the first amount of torque applied to the first rotatable knob may be required to rotate the actuation mechanism relative to the distal shell portion in a second rotational direction.

In addition, or alternatively, to any example disclosed herein, the actuation mechanism comprises a longitudinal slot formed in an outer surface of the actuation mechanism proximate a distal end of the actuation mechanism.

In addition, or alternatively, to any example disclosed herein, a radial depth of the longitudinal slot is tapered in a circumferential direction.

In addition, or alternatively, to any example disclosed herein, the radial depth of the longitudinal slot is greater at a second end of the longitudinal slot than at a first end of the longitudinal slot.

In addition, or alternatively, to any example disclosed herein, the friction element is disposed within the longitudinal slot.

In addition, or alternatively, to any example disclosed herein, the friction element extends radially outward of the outer surface of the actuation mechanism.

In addition, or alternatively, to any example disclosed herein, the friction element comprises an elastomeric rod.

In addition, or alternatively, to any example disclosed herein, the friction element has a circular cross-section.

In addition, or alternatively, to any example disclosed herein, rotation of the actuation mechanism in the first rotational direction is configured to axially translate the first portion of the elongate shaft assembly distally relative to the handle assembly, and rotation of the actuation mechanism in the second rotational direction is configured to axially translate the first portion of the elongate shaft assembly proximally relative to the handle assembly.

In addition, or alternatively, to any example disclosed herein, the handle assembly comprises a second rotatable knob configured to rotate around the central longitudinal axis of the handle assembly to axially translate a second portion of the elongate shaft assembly along the central longitudinal axis.

In addition, or alternatively, to any example disclosed herein, rotation of the second rotatable knob in the first rotational direction is configured to axially translate the second portion of the elongate shaft assembly proximally relative to the handle assembly, and rotation of the second rotatable knob in the second rotational direction is configured to axially translate the second portion of the elongate shaft assembly distally relative to the handle assembly.

In addition, or alternatively, to any example disclosed herein, a delivery device for delivering a replacement heart valve implant to a native heart valve may comprise a handle assembly and an elongate shaft assembly extending distally from the handle assembly. The handle assembly may comprise a first rotatable knob configured to rotate around a central longitudinal axis of the handle assembly, an actuation mechanism operatively engaged with the first rotatable knob, a distal shell portion disposed radially outward of the actuation mechanism, and a plurality of friction elements engaged with the actuation mechanism and the distal shell portion. The first rotatable knob may be configured to rotate the actuation mechanism relative to the distal shell portion to axially translate a portion of the elongate shaft assembly along the central longitudinal axis. A first amount of torque applied to the first rotatable knob may be required to rotate the actuation mechanism relative to the distal shell portion in a first rotational direction, and a second amount of torque applied to the first rotatable knob greater than the first amount of torque applied to the first rotatable knob may be required to rotate the actuation mechanism relative to the distal shell portion in a second rotational direction.

In addition, or alternatively, to any example disclosed herein, the actuation mechanism comprises a plurality of longitudinal slots formed in an outer surface of the actuation mechanism proximate a distal end of the actuation mechanism.

In addition, or alternatively, to any example disclosed herein, each friction element of the plurality of friction elements is disposed within a separate longitudinal slot of the plurality of longitudinal slots.

In addition, or alternatively, to any example disclosed herein, delivery device for delivering a replacement heart valve implant to a native heart valve may comprise a handle assembly and an elongate shaft assembly extending distally from the handle assembly. The handle assembly may comprise a first rotatable knob configured to rotate around a central longitudinal axis of the handle assembly, an actuation mechanism operatively engaged with the first rotatable knob, a distal shell portion disposed radially outward of the actuation mechanism, and a friction element disposed within a longitudinal slot formed in an outer surface of the actuation mechanism, wherein the friction element may be engaged in compression between the actuation mechanism and the distal shell portion. The first rotatable knob may be configured to rotate the actuation mechanism relative to the distal shell portion to axially translate a first portion of the elongate shaft assembly along the central longitudinal axis. The friction element may be engaged between the actuation mechanism and the distal shell portion in a first amount of compression when the actuation mechanism is rotated relative to the distal shell portion in a first rotational direction, and the friction element may be engaged between the actuation mechanism and the distal shell portion in a second amount of compression greater than the first amount of compression when the actuation mechanism is rotated relative to the distal shell portion in a second rotational direction.

In addition, or alternatively, to any example disclosed herein, the first amount of compression is at least 1% and the second amount of compression is at least 10%.

In addition, or alternatively, to any example disclosed herein, a first amount of torque applied to the first rotatable knob is required to rotate the actuation mechanism relative to the distal shell portion in the first rotational direction, and a second amount of torque applied to the first rotatable knob greater than the first amount of torque applied to the first rotatable knob is required to rotate the actuation mechanism relative to the distal shell portion in the second rotational direction.

In addition, or alternatively, to any example disclosed herein, the second amount of torque is at least 200% greater than the first amount of torque.

In addition, or alternatively, to any example disclosed herein, the friction element comprises an elastomeric rod having a circular cross-section defining an outer diameter of the elastomeric rod.

In addition, or alternatively, to any example disclosed herein, the longitudinal slot comprises a first circumferential wall and a second circumferential wall collectively defining a circumferential length of the longitudinal slot at an outer perimeter of the actuation mechanism. The circumferential length of the longitudinal slot at the outer perimeter of the actuation mechanism is greater than 110% of the outer diameter of the elastomeric rod and less than 140% of the outer diameter of the elastomeric rod.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically illustrates selected aspects of a replacement heart valve implant;

FIGS. 2-3 schematically illustrate selected aspects of a replacement heart valve system including the replacement heart valve implant and a delivery device;

FIGS. 4-5 are partial cross-sectional views illustrating selected aspects of a handle assembly of the delivery device;

FIG. 6 is a partial cutaway view illustrating selected aspects of the handle assembly of the delivery device of FIGS. 4-5;

FIG. 7 is a partial cross-sectional view taken along the line 7-7 of FIG. 5;

FIG. 8 is a detailed view of a portion of the handle assembly shown in FIG. 7; and

FIGS. 9-10 are partial cross-sectional views schematically illustrating selected aspects related to the function of the handle assembly.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently - such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

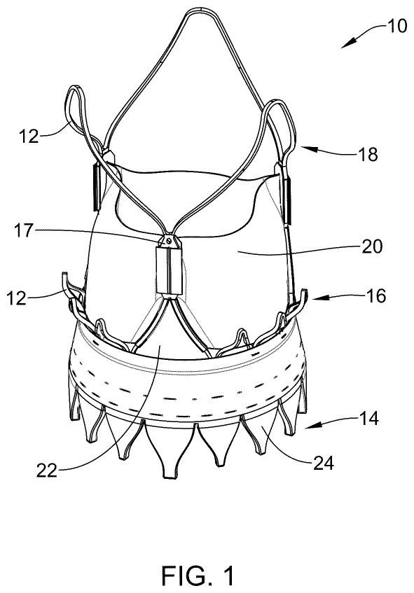

FIG. 1 illustrates selected aspects of a replacement heart valve implant 10. It should be appreciated that the replacement heart valve implant 10 can be any type of replacement heart valve (e.g., a mitral valve, an aortic valve, etc.). Some non-limiting examples of the replacement heart valve implant 10 may include the ACURATE NEO2™, the ACURATE PRIME™, and/or family members thereof from Boston Scientific. Other examples are also contemplated. In use, the replacement heart valve implant 10 may be implanted (e.g., surgically or through transcatheter delivery) within a native heart valve in a mammalian heart. The replacement heart valve implant 10 can be configured to allow one-way flow through the replacement heart valve implant 10 from an inflow end to an outflow end.

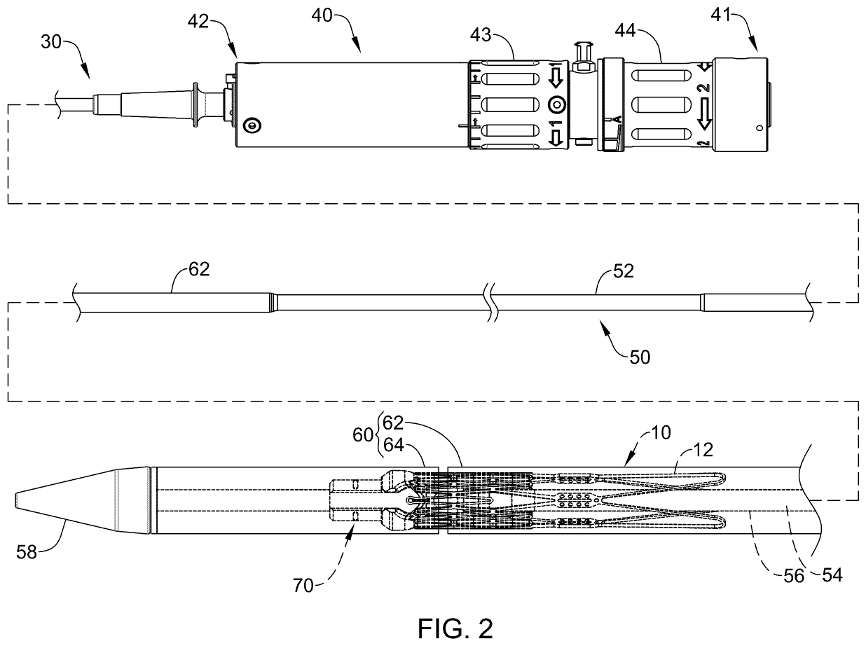

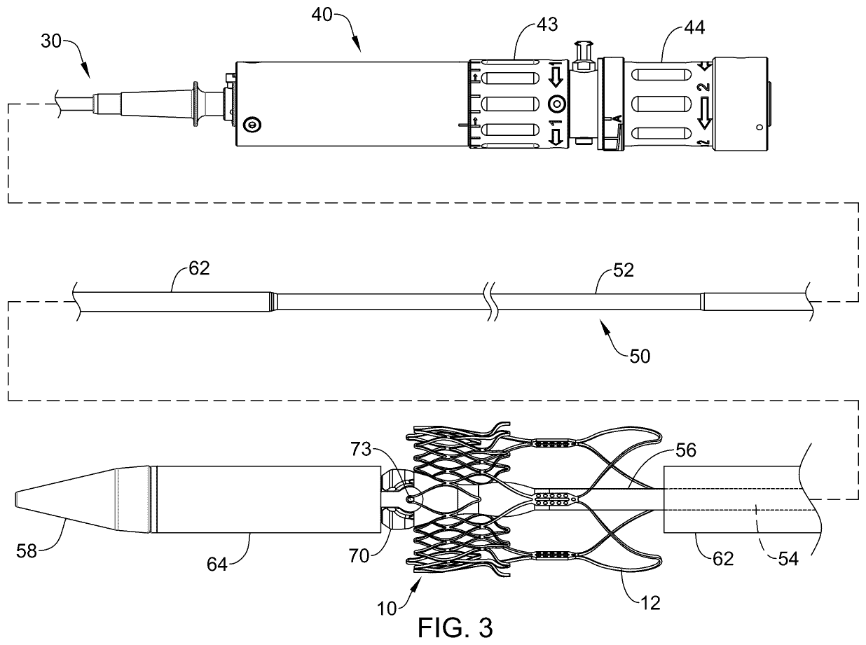

The replacement heart valve implant 10 may include an expandable framework 12 defining a central lumen. In some embodiments, the expandable framework 12 may have a substantially circular cross-section. In some embodiments, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The replacement heart valve implant 10 and/or the expandable framework 12 may be configured to shift between a radially collapsed configuration (e.g., FIG. 2) and a radially expanded configuration (e.g., FIG. 3). In some embodiments, the expandable framework 12 may be self-expanding. In some embodiments, the expandable framework 12 may be self-biased toward the radially expanded configuration. In some embodiments, the expandable framework 12 may be mechanically expandable. In some embodiments, the expandable framework 12 may be balloon expandable. Other configurations, including combinations thereof, are also contemplated.

In some embodiments, the expandable framework 12 may define a lower crown 14 proximate and/or at an inflow end, an upper crown 16 proximate and/or at an outflow end, and a plurality of stabilization arches 18 extending downstream from the outflow end. The expandable framework may comprise a body portion extending between the lower crown 14 and the upper crown 16. In at least some embodiments, the body portion may be formed as and/or similar to a stent or a stent-like structure. In some embodiments, the body portion may comprise a plurality of closed cells. In some embodiments, the expandable framework 12 may comprise a plurality of commissure posts 17. In some embodiments, the plurality of commissure posts 17 may extend downstream of and/or away from the upper crown 16. In some embodiments, the plurality of stabilization arches 18 may extend downstream of and/or away from the upper crown 16 and/or the plurality of commissure posts 17 in a direction opposite the lower crown 14. In some embodiments, the upper crown 16 and/or the plurality of commissure posts 17 may be disposed longitudinally and/or axially between the lower crown 14 and the plurality of stabilization arches 18.

In some embodiments, the replacement heart valve implant 10 and/or the expandable framework 12 may include a proximal portion and a distal portion. In some embodiments, orientation of the replacement heart valve implant 10 may be related to a delivery device 30 (e.g., FIGS. 2-3) and/or a direction of implantation relative to a treatment site (e.g., a native heart valve). In some embodiments, the proximal portion may include the outflow end and/or the plurality of stabilization arches 18. In some embodiments, the proximal portion may include the upper crown 16 and/or the plurality of commissure posts 17. In some embodiments, the distal portion may include the inflow end and/or the lower crown 14. In some embodiments, the distal portion may include the body portion and/or a portion of the body portion disposed immediately adjacent the lower crown 14. Other configurations are also contemplated.

In some embodiments, the replacement heart valve implant 10 may include a plurality of valve leaflets 20 disposed within the central lumen. The plurality of valve leaflets 20 may be coupled, secured, and/or fixedly attached to the expandable framework 12 at the plurality of commissure posts 17 to form and/or define a plurality of commissures. In addition, or alternatively, in some embodiments, the plurality of valve leaflets 20 may be coupled, secured, and/or fixedly attached to the expandable framework 12 proximate and/or at other locations, such as the inflow end, the lower crown 14, etc. The plurality of valve leaflets 20 may be configured to shift between an open position and a closed position. The plurality of valve leaflets 20 may be configured to substantially restrict fluid flow through the replacement heart valve implant 10 in the closed position. The plurality of valve leaflets 20 may move apart from each other and/or radially outward within the central lumen in the open position to permit fluid flow through the replacement heart valve implant 10 and/or the central lumen.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from porcine pericardium, bovine pericardium, or other tissue. Other configurations and/or materials are also contemplated.

In some embodiments, the replacement heart valve implant 10 may include an inner skirt 22 disposed on and/or extending along an inner surface of the expandable framework 12. In at least some embodiments, the inner skirt 22 may be fixedly attached to the expandable framework 12. The inner skirt 22 may direct fluid, such as blood, flowing through the replacement heart valve implant 10 toward the plurality of valve leaflets 20. In at least some embodiments, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 may include an outer skirt 24 disposed on and/or extending along an outer surface of the expandable framework 12. In some embodiments, the outer skirt 24 may be disposed at and/or adjacent the lower crown 14. The outer skirt 24 may ensure the fluid flows through the replacement heart valve implant 10 and does not flow around the replacement heart valve implant 10 (e.g., between the expandable framework 12 and the vessel wall).

In some embodiments, the inner skirt 22 and/or the outer skirt 24 may include a polymer, and/or may include at least 50 percent by weight of a polymer. In some embodiments, the inner skirt 22 and/or the outer skirt 24 may be substantially impervious to fluid. In some embodiments, the inner skirt 22 and/or the outer skirt 24 may be formed from a thin tissue (e.g., porcine pericardium, bovine pericardium, or other tissue, etc.), a coated fabric material, or a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, a plurality of, or each of a plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 using one or more methods including but not limited to tying with sutures or filaments, adhesive bonding, melt bonding, embedding or over molding, welding, etc.

In some embodiments, the expandable framework 12 and/or the replacement heart valve implant 10 may have an outer extent of about 17 mm (mm) (0.669 inches), about 19.5 mm (0.768 inches), about 23 millimeters (0.905 inches), about 25 mm (0.984 inches), about 27 mm (1.063 inches), about 30 mm (1.181 inches), about 31 mm (1.220 inches), etc. in an unconstrained configuration (e.g., in the radially expanded configuration). In some embodiments, the expandable framework 12 and/or the replacement heart valve implant 10 may have an outer extent of about 11 mm (0.433 inches), about 10 mm (0.394 inches), about 9 mm (0.354 inches), about 8 mm (0.315 inches), about 7 mm (0.276 inches), about 6 mm (0.236 inches), about 5 mm (0.197 inches), about 4 mm (0.157 inches), etc. in the radially collapsed configuration. Other configurations are also contemplated.

FIGS. 2-3 illustrate selected aspects of a replacement heart valve system comprising the replacement heart valve implant 10 and a delivery device 30 for delivering the replacement heart valve implant 10 to a native heart valve. It should be noted that FIGS. 2-3 include at least one change of scale (e.g., all parts of the figure are not drawn to the same scale) to improve viewability and show additional detail of selected aspects of the delivery device 30. Additionally, the expandable framework 12 is shown and some elements of the replacement heart valve implant 10 are omitted from FIGS. 2-3 to improve clarity. It should further be noted that all elements are not shown or labelled in each figure in the interest of clarity.

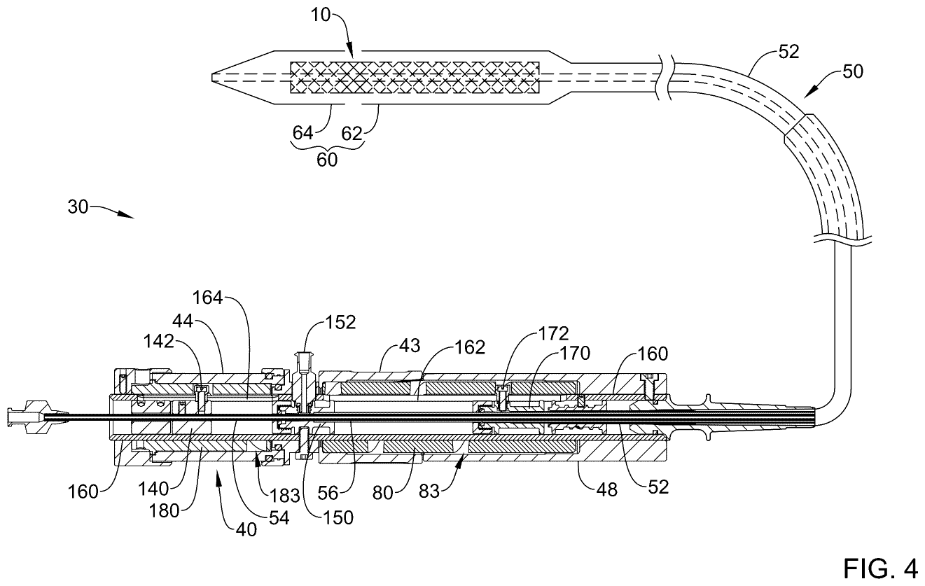

The delivery device 30 may include a handle assembly 40 and an elongate shaft assembly 50 extending distally from the handle assembly 40. The handle assembly 40 may include a proximal end 41 and a distal end 42 opposite the proximal end 41. The elongate shaft assembly 50 may extend distally from the distal end 42 of the handle assembly 40. Some aspects related to the construction and function of the handle assembly 40 are shown in more detail in FIGS. 4-7. In some embodiments, the handle assembly 40 may include one or more rotatable knobs. In some embodiments, the one or more rotatable knobs may include a first rotatable knob 43 and a second rotatable knob 44. In at least some embodiments, the first rotatable knob 43 and/or the second rotatable knob 44 may be configured to rotate around a central longitudinal axis of the delivery device 30 and/or the handle assembly 40. Other configurations and/or types of interface controls are also contemplated.

In some embodiments, rotation of the first rotatable knob 43 around the central longitudinal axis of the handle assembly 40 may be configured to deploy the proximal portion of the replacement heart valve implant 10 from an implant holding portion 60 of the delivery device 30. Similarly, rotation of the second rotatable knob 44 around the central longitudinal axis of the handle assembly 40 may be configured to deploy the distal portion of the replacement heart valve implant 10 from the implant holding portion 60 of the delivery device 30.

In some embodiments, a distal portion of the delivery device 30 and/or the elongate shaft assembly 50 may comprise the implant holding portion 60. The implant holding portion 60 may be configured to engage with and/or constrain the replacement heart valve implant 10 and/or the expandable framework 12 in the radially collapsed configuration. The elongate shaft assembly 50 may include an outer tubular member 52 extending distally from the handle assembly 40 and an inner shaft 54 extending distally from the handle assembly 40 within the outer tubular member 52 to a distal tip 58 disposed distal of the implant holding portion 60. In some embodiments, the implant holding portion 60 may comprise a proximal sheath 62 and a distal sheath 64. In some embodiments, the proximal sheath 62 and/or the distal sheath 64 may be formed from a polymeric material. In some embodiments, the proximal sheath 62 and/or the distal sheath 64 may include a reinforcing structure disposed therein and/or thereon. In some embodiments, the reinforcing structure may be a coil, a mesh, one or more filaments, bands, or strips, or another suitable structure. Other configurations are also contemplated.

In some embodiments, the inner shaft 54 may be slidably disposed within a lumen of the outer tubular member 52. In some embodiments, the elongate shaft assembly 50 may include an intermediate tubular member 56 disposed within and/or radially inward of the outer tubular member 52 and about and/or radially outward of the inner shaft 54. In at least some embodiments, the inner shaft 54 and the outer tubular member 52 are each axially translatable relative to the intermediate tubular member 56 independently of each other. For example, the inner shaft 54 may be translated relative to the intermediate tubular member 56 without translating the outer tubular member 52 relative to the intermediate tubular member 56, and vice versa.

In some embodiments, the proximal sheath 62 may be fixedly attached to the outer tubular member 52. In some embodiments, the proximal sheath 62 may be fixedly attached to and/or may extend distally from a distal end of the outer tubular member 52. In some embodiments, the distal sheath 64 and/or the distal tip 58 may be fixedly attached to the inner shaft 54. In some embodiments, the distal sheath 64 may be fixedly attached to the distal tip 58. In some embodiments, the distal sheath 64 may extend proximally from the distal tip 58. In some embodiments, the inner shaft 54 may include and/or at least partially define a guidewire lumen extending therethrough. In some embodiments, the guidewire lumen may extend through the handle assembly 40.

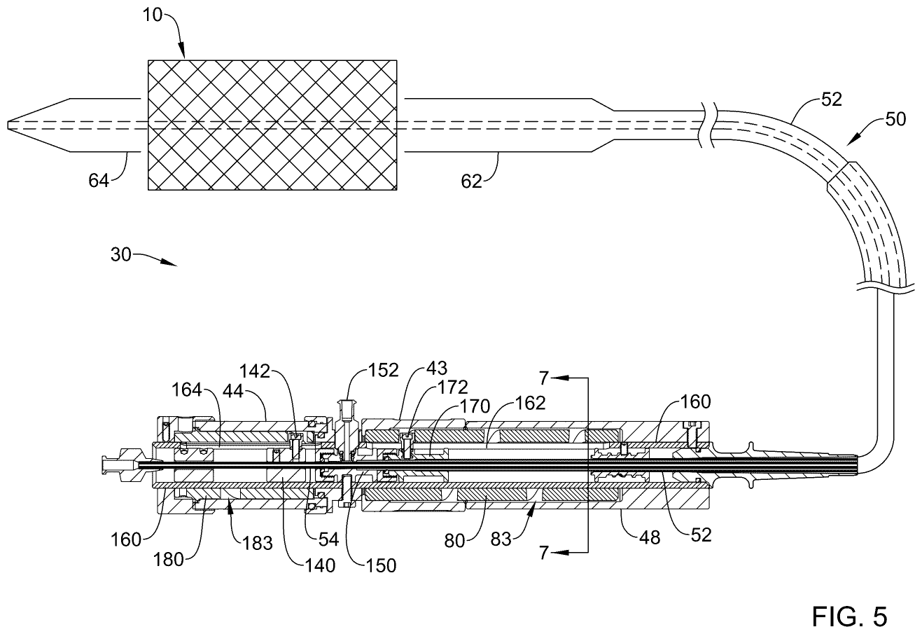

In some embodiments, the handle assembly 40 may be configured to manipulate and/or translate the proximal sheath 62 and/or the distal sheath 64 relative to each other using the first rotatable knob 43 and/or the second rotatable knob 44, as discussed further below, to shift the implant holding portion 60 between a closed configuration (e.g., FIGS. 2, 4) and an open configuration (e.g., FIGS. 3, 5). In some embodiments, the handle assembly 40 and/or the second rotatable knob 44 may be configured to manipulate and/or translate the inner shaft 54 and/or the distal sheath 64 relative to the elongate shaft assembly 50, the outer tubular member 52, the intermediate tubular member 56, and/or the proximal sheath 62. In some embodiments, the handle assembly 40 and/or the first rotatable knob 43 may be configured to manipulate and/or translate the outer tubular member 52 and/or the proximal sheath 62 relative to the elongate shaft assembly 50, the inner shaft 54, the intermediate tubular member 56, and/or the distal sheath 64.

During delivery of the replacement heart valve implant 10 to a treatment site (e.g., the native heart valve, the aortic valve, etc.), the replacement heart valve implant 10 may be disposed at least partially within the proximal sheath 62 and/or the distal sheath 64 in the radially collapsed configuration in the closed configuration of the implant holding portion 60. In some embodiments, the proximal sheath 62 and/or the distal sheath 64 may collectively define the implant holding portion 60 of the delivery device 30. In some embodiments, the implant holding portion 60 may be configured to constrain the replacement heart valve implant 10 in the radially collapsed configuration when the implant holding portion 60 is in the closed configuration. In some embodiments, the replacement heart valve implant 10 may be releasably coupled to and/or releasably engaged with the intermediate tubular member 56 and/or a stent holder 70 when the replacement heart valve implant 10 is constrained within the implant holding portion 60 of the delivery device 30 in the radially collapsed configuration.

In some embodiments, the stent holder 70 may be fixedly attached to the elongate shaft assembly 50. In some embodiments, the stent holder 70 may be fixedly attached to the intermediate tubular member 56 of the elongate shaft assembly 50. In some alternative embodiments, the stent holder 70 may be monolithically formed with the elongate shaft assembly 50 and/or the intermediate tubular member 56. In some embodiments, the stent holder 70 may be configured to engage the expandable framework 12 in the radially collapsed configuration and/or when the replacement heart valve implant 10 is constrained within the implant holding portion 60 of the delivery device 30. In some embodiments, the stent holder 70 may include at least one projection 73 (e.g., FIG. 3) configured to engage the expandable framework 12 and/or the lower crown 14 in the radially collapsed configuration.

In some embodiments, the proximal sheath 62 may be configured to cover the proximal portion of the replacement heart valve implant 10 in the radially collapsed configuration when the implant holding portion 60 is in the closed configuration, and the distal sheath 64 may be configured to cover the distal portion of the replacement heart valve implant 10 in the radially collapsed configuration when the implant holding portion 60 is in the closed configuration. In some embodiments, the replacement heart valve implant 10 may be constrained in the radially collapsed configuration by the proximal sheath 62 and the distal sheath 64 in the closed configuration of the implant holding portion 60. In some embodiments, the proximal sheath 62 may be disposed adjacent to the distal sheath 64 in the closed configuration. In some embodiments, the proximal sheath 62 may abut the distal sheath 64 in the closed configuration. In some embodiments, the proximal sheath 62 may be axially spaced apart from the distal sheath 64 in the closed configuration. In some embodiments, the proximal sheath 62 may be axially spaced apart from the distal sheath 64 in the closed configuration by less than 20% of an overall length of the replacement heart valve implant 10 in the radially collapsed configuration. In some embodiments, the proximal sheath 62 may be axially spaced apart from the distal sheath 64 in the closed configuration by less than 15% of an overall length of the replacement heart valve implant 10 in the radially collapsed configuration. In some embodiments, the proximal sheath 62 may be axially spaced apart from the distal sheath 64 in the closed configuration by less than 10% of an overall length of the replacement heart valve implant 10 in the radially collapsed configuration. In some embodiments, the proximal sheath 62 may be axially spaced apart from the distal sheath 64 in the closed configuration by less than 5% of an overall length of the replacement heart valve implant 10 in the radially collapsed configuration. Other configurations are also contemplated.

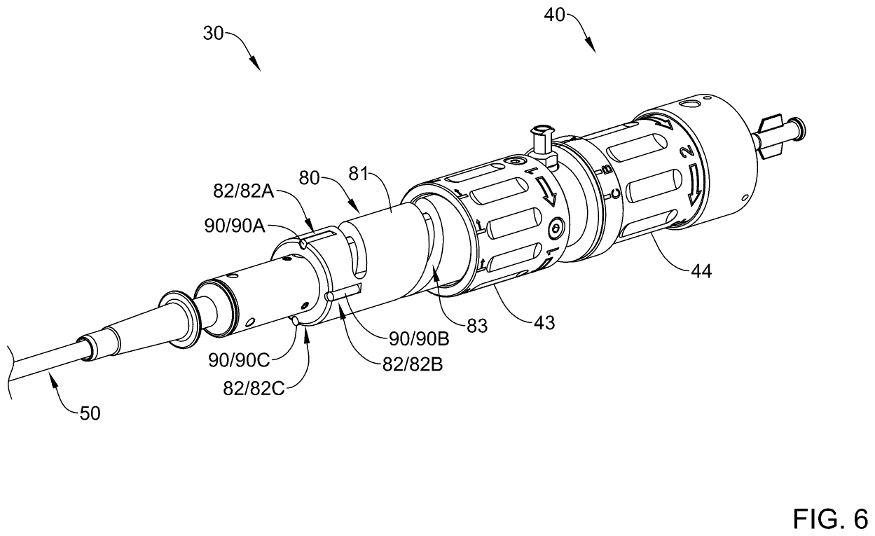

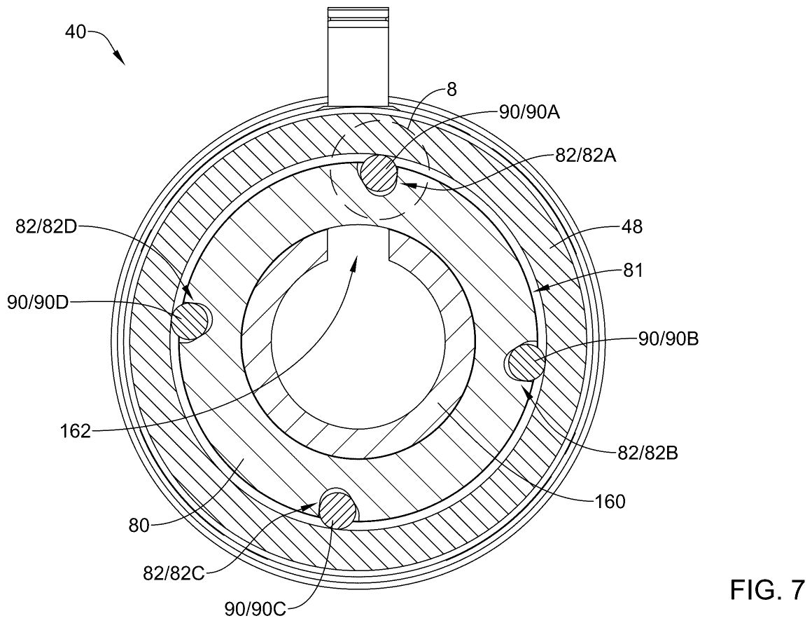

FIGS. 4-5 are partial cross-sectional views illustrating selected aspects of the handle assembly 40 in more detail. Additional aspects of the handle assembly 40 may be seen in FIGS. 6-7. Some features and/or elements are shown schematically or are omitted to improve clarity. For example, the replacement heart valve implant 10 and the implant holding portion 60 are illustrated schematically.

In some embodiments, the handle assembly 40 may comprise a guide tube 160 disposed therein. In some embodiments, the guide tube 160 may include a lumen extending therein. The guide tube 160 may comprise a distal longitudinal slot 162 formed through a wall of the guide tube 160 in a distal portion of the guide tube 160. The guide tube 160 may comprise a proximal longitudinal slot 164 formed through the wall of the guide tube 160 in a proximal portion of the guide tube 160.

In some embodiments, the handle assembly 40 may comprise a proximal hub 170 fixedly attached to a proximal end of the outer tubular member 52. The proximal hub 170 may be disposed within and selectively movable axially relative to the guide tube 160. In some embodiments, the proximal hub 170 may be disposed within the lumen of the guide tube 160. The proximal hub 170 may be disposed within the distal portion of the guide tube 160. The proximal hub 170 may include a guide member 172 extending radially outward from the proximal hub 170. In some embodiments, the proximal hub 170 may include a proximal flange disposed proximate a proximal end of the proximal hub 170 and/or a distal flange disposed proximate a distal end of the proximal hub 170. In some embodiments, the proximal flange and/or the distal flange may be configured to slide along the wall of the guide tube 160. In some embodiments, the proximal hub 170 may be overmolded onto the proximal end of the outer tubular member 52. In some embodiments, the proximal hub 170 may be formed separately from the outer tubular member 52 and later fixedly attached to the proximal end of the outer tubular member 52, such as by adhesive bonding, welding, friction and/or interference fit, mechanical attachment, etc.

In some embodiments, the handle assembly 40 may comprise a distal shell portion 48 disposed proximate the distal end 42 of the handle assembly 40. The distal shell portion 48 may be substantially hollow and/or may be configured to cover and/or protect movable parts disposed therein. The distal shell portion 48 may be disposed distal of the first rotatable knob 43. In some embodiments, the distal shell portion 48 may form and/or may be usable as a hand grip for a user of the delivery device 30.

In some embodiments, the handle assembly 40 comprises an actuation mechanism 80 operatively engaged with the first rotatable knob 43. In some embodiments, the first rotatable knob 43 may be fixedly attached to the actuation mechanism 80. In some embodiments, the actuation mechanism 80 may be a tubular structure surrounding and/or coaxial with the central longitudinal axis of the handle assembly 40. The actuation mechanism 80 may comprise an outer surface 81 (e.g., FIG. 6). In some embodiments, at least a portion of the distal shell portion 48 may be disposed radially outward of at least a portion of the actuation mechanism 80. In FIG. 6, which is a partial cutaway view illustrating selected aspects of the handle assembly of the delivery device of FIGS. 4-5, the distal shell portion 48 has been removed to show some aspects of the actuation mechanism 80 and/or the handle assembly 40. In some embodiments, the actuation mechanism 80 may comprise a longitudinal slot 82 formed in the outer surface 81 of the actuation mechanism 80 proximate a distal end of the actuation mechanism 80. In some embodiments, the actuation mechanism 80 may comprise a plurality of longitudinal slots (refs. 82A, 82B, 82C, 82D—e.g., FIG. 7) formed in the outer surface 81 of the actuation mechanism 80 proximate the distal end of the actuation mechanism 80, seen in FIGS. 6-7.

In some embodiments, the handle assembly 40 may comprise a friction element 90 engaged with the actuation mechanism 80 and the distal shell portion 48. In some embodiments, the friction element 90 may be disposed within the longitudinal slot 82. The friction element 90 may extend radially outward of the outer surface 81 of the actuation mechanism 80. In some embodiments, the friction element 90 may comprise an elastomeric rod. In some embodiments, the friction element 90 and/or the elastomeric rod may have a substantially circular cross-section defining an outer diameter of the friction element 90 and/or the elastomeric rod in a neutral state, a natural state, and/or when unstressed. Other configurations are also contemplated.

In some embodiments, the handle assembly 40 may comprise a plurality of friction elements (refs. 90A, 90B, 90C, 90D—e.g., FIG. 7) engaged with the actuation mechanism 80 and the distal shell portion 48. In some embodiments, the plurality of friction elements (refs. 90A, 90B, 90C, 90D—e.g., FIG. 7) may be disposed within the plurality of longitudinal slots (refs. 82A, 82B, 82C, 82D—e.g., FIG. 7). In some embodiments, each friction element of plurality of friction elements (refs. 90A, 90B, 90C, 90D—e.g., FIG. 7) may be disposed within a separate longitudinal slot of the plurality of longitudinal slots (refs. 82A, 82B, 82C, 82D—e.g., FIG. 7). The plurality of friction elements (refs. 90A, 90B, 90C, 90D—e.g., FIG. 7) may extend radially outward of the outer surface 81 of the actuation mechanism 80. In some embodiments, the plurality of friction elements (refs. 90A, 90B, 90C, 90D—e.g., FIG. 7) may each comprise an elastomeric rod. In some embodiments, the plurality of friction elements (refs. 90A, 90B, 90C, 90D—e.g., FIG. 7) may each have a substantially circular cross-section defining an outer diameter thereof in a neutral state, a natural state, and/or when unstressed. Other configurations are also contemplated.

The actuation mechanism 80 may comprise a first helical guide 83 extending radially through the actuation mechanism 80. The first helical guide 83 may be configured to engage with and/or interact with the guide member 172. For example, the guide member 172 may be disposed within the first helical guide 83, as seen in FIGS. 4-5. Additionally, at least a portion of the guide member 172 may be disposed within the distal longitudinal slot 162. The distal longitudinal slot 162 may be configured to prevent rotation of the guide member 172 and/or the proximal hub 170 relative to the central longitudinal axis of the handle assembly 40.

In some embodiments, the first rotatable knob 43 may be configured to rotate the actuation mechanism 80 relative to the central longitudinal axis of the handle assembly 40 and/or the distal shell portion 48. In some embodiments, rotation of the first rotatable knob 43 around the central longitudinal axis of the handle assembly 40 causes rotation of and/or rotates the actuation mechanism 80 around the central longitudinal axis of the handle assembly 40 within the distal shell portion 48.

In some embodiments, the first rotatable knob 43 may be configured to rotate the actuation mechanism 80 relative to the central longitudinal axis of the handle assembly 40 and/or the distal shell portion 48 of the handle assembly 40 to axially translate the proximal hub 170 within the guide tube 160 and/or to axially translate a first portion of the elongate shaft assembly 50 along the central longitudinal axis. In some embodiments, the first portion of the elongate shaft assembly 50 may comprise and/or may be the outer tubular member 52 and/or the proximal sheath 62. In some embodiments, rotation of the first rotatable knob 43 and/or the actuation mechanism 80 may be configured to axially translate the first portion (e.g., the outer tubular member 52 and/or the proximal sheath 62) of the elongate shaft assembly 50 relative to the handle assembly 40. In some embodiments, rotation of the first rotatable knob 43 and/or the actuation mechanism 80 in a first rotational direction may be configured to axially translate the first portion (e.g., the outer tubular member 52 and/or the proximal sheath 62) of the elongate shaft assembly 50 distally relative to the handle assembly 40 and/or toward the closed configuration of the implant holding portion 60 (e.g., FIG. 4). In some embodiments, rotation of the first rotatable knob 43 and/or the actuation mechanism 80 in a second rotational direction (e.g., opposite the first rotational direction) may be configured to axially translate the first portion (e.g., the outer tubular member 52 and/or the proximal sheath 62) of the elongate shaft assembly 50 proximally relative to the handle assembly 40 and/or toward the open configuration of the implant holding portion 60 (e.g., FIG. 5). In at least some embodiments, the first rotational direction may be clockwise as viewed from the proximal end 41 of the handle assembly 40 to the distal end 42 of the handle assembly 40, and the second rotational direction may be counterclockwise as viewed from the proximal end 41 of the handle assembly 40 to the distal end 42 of the handle assembly 40.

In some embodiments, the handle assembly 40 may comprise a flush hub 150. In some embodiments, the flush hub 150 may be disposed between the first rotatable knob 43 and the second rotatable knob 44. Other configurations are also contemplated. In some embodiments, the flush hub 150 may comprise a flush port 152. In some embodiments, the flush port 152 may be used to remove air from the delivery device 30 prior to performing a procedure. Other configurations are also contemplated. In some embodiments, the intermediate tubular member 56 of the elongate shaft assembly 50 may be fixedly attached and/or fixedly secured to the flush hub 150 such that the intermediate tubular member 56 is axially fixed relative to the handle assembly 40.

In some embodiments, the handle assembly 40 may comprise a slide block 140 slidably disposed within the proximal portion of the guide tube 160. The inner shaft 54 of the elongate shaft assembly 50 may be fixedly secured to the slide block 140. In some embodiments, the inner shaft 54 of the elongate shaft assembly 50 may be fixedly secured to the slide block 140 using a locking element such as a set screw, a pin, etc. In some embodiments, the slide block 140 may comprise a second guide member 142 extending radially outward from the slide block 140.

In some embodiments, the handle assembly 40 may comprise a second actuation mechanism 180 operatively engaged with the second rotatable knob 44. In some embodiments, the second rotatable knob 44 may be fixedly attached to the second actuation mechanism 180. In some embodiments, the second actuation mechanism 180 may be a tubular structure surrounding and/or coaxial with the central longitudinal axis of the handle assembly 40.

The second actuation mechanism 180 may comprise a second helical guide 183 extending radially through the second actuation mechanism 180. The second helical guide 183 may be configured to engage with and/or interact with the second guide member 142. For example, the second guide member 142 may be disposed within the second helical guide 183. Additionally, at least a portion of the second guide member 142 may be disposed within the proximal longitudinal slot 164. The proximal longitudinal slot 164 may be configured to prevent rotation of the second guide member 142 and/or the slide block 140 relative to the central longitudinal axis of the handle assembly 40.

In some embodiments, the second rotatable knob 44 may be configured to rotate the second actuation mechanism 180 relative to the central longitudinal axis of the handle assembly 40. In some embodiments, rotation of the second rotatable knob 44 around the central longitudinal axis of the handle assembly 40 causes rotation of and/or rotates the second actuation mechanism 180 around the central longitudinal axis of the handle assembly 40.

In some embodiments, the second rotatable knob 44 may be configured to rotate the second actuation mechanism 180 relative to the central longitudinal axis of the handle assembly 40 to axially translate the slide block 140 within the guide tube 160 and/or to axially translate a second portion of the elongate shaft assembly 50 along the central longitudinal axis. In some embodiments, the second portion of the elongate shaft assembly 50 may comprise and/or may be the inner shaft 54 and/or the distal sheath 64. In some embodiments, rotation of the second rotatable knob 44 and/or the second actuation mechanism 180 may be configured to axially translate the second portion (e.g., the inner shaft 54 and/or the distal sheath 64) of the elongate shaft assembly 50 relative to the handle assembly 40. In some embodiments, rotation of the second rotatable knob 44 and/or the second actuation mechanism 180 in the first rotational direction may be configured to axially translate the second portion (e.g., the inner shaft 54 and/or the distal sheath 64) of the elongate shaft assembly 50 proximally relative to the handle assembly 40 and/or toward the closed configuration of the implant holding portion 60 (e.g., FIG. 4). In some embodiments, rotation of the second rotatable knob 44 and/or the second actuation mechanism 180 in the second rotational direction (e.g., opposite the first rotational direction) may be configured to axially translate the second portion (e.g., the inner shaft 54 and/or the distal sheath 64) of the elongate shaft assembly 50 distally relative to the handle assembly 40 and/or toward the open configuration of the implant holding portion 60 (e.g., FIG. 5).

FIG. 7 is a partial cross-sectional view taken along the line 7-7 in FIG. 5 as though FIG. 5 were not in partial cross-section (e.g., FIG. 7 does not illustrate a cross-section of a cross-section). For improved clarity, some features and/or elements are omitted. FIG. 7 illustrates in additional detail some aspects previously described herein, and thus that description is not repeated.

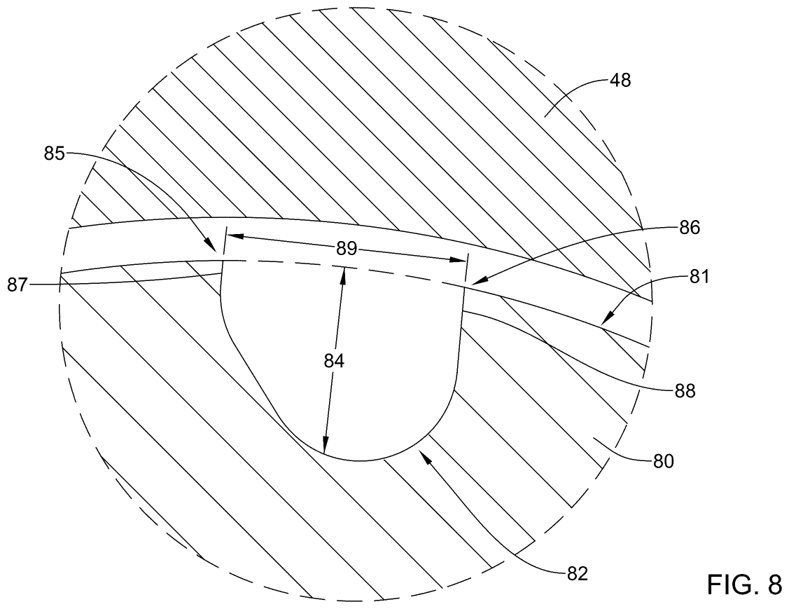

FIG. 8 is a detail view of the longitudinal slot 82 circled in FIG. 7 and identified by reference number 8. As may be appreciated, all details related to the longitudinal slot 82 of FIG. 8 may apply equally to any and/or each longitudinal slot of the plurality of longitudinal slots (refs. 82A, 82B, 82C, 82D—e.g., FIG. 7). In some embodiments, a radial depth 84 of the longitudinal slot 82 and/or the plurality of longitudinal slots (refs. 82A, 82B, 82C, 82D—e.g., FIG. 7) may be tapered in a circumferential direction. In some embodiments, the radial depth 84 of the longitudinal slot 82 and/or the plurality of longitudinal slots (refs. 82A, 82B, 82C, 82D—e.g., FIG. 7) may be greater at a second end 86 of the longitudinal slot 82 and/or the plurality of longitudinal slots (refs. 82A, 82B, 82C, 82D—e.g., FIG. 7), as viewed in a distal to proximal direction along the central longitudinal axis, than at a first end 85 of the longitudinal slot 82 and/or the plurality of longitudinal slots (refs. 82A, 82B, 82C, 82D—e.g., FIG. 7).

In some embodiments, the longitudinal slot 82 and/or the plurality of longitudinal slots (refs. 82A, 82B, 82C, 82D—e.g., FIG. 7) may comprise a first circumferential wall 87 disposed at the first end 85 of the longitudinal slot 82 and/or the plurality of longitudinal slots (refs. 82A, 82B, 82C, 82D—e.g., FIG. 7) and a second circumferential wall 88 disposed at the second end 86 of the longitudinal slot 82 and/or the plurality of longitudinal slots (refs. 82A, 82B, 82C, 82D—e.g., FIG. 7), the first circumferential wall 87 and the second circumferential wall 88 collectively defining a circumferential length 89 of the longitudinal slot 82 and/or the plurality of longitudinal slots (refs. 82A, 82B, 82C, 82D—e.g., FIG. 7).

In some embodiments, the circumferential length 89 of the longitudinal slot 82 and/or the plurality of longitudinal slots (refs. 82A, 82B, 82C, 82D—e.g., FIG. 7) at an outer perimeter of the actuation mechanism 80 may be greater than 110% of the outer diameter of the friction element 90 and/or the elastomeric rod disposed within the longitudinal slot 82 and/or the plurality of friction elements (refs. 90A, 90B, 90C, 90D—e.g., FIG. 7) and/or the elastomeric rods disposed within the plurality of longitudinal slots (refs. 82A, 82B, 82C, 82D—e.g., FIG. 7). In some embodiments, the circumferential length 89 of the longitudinal slot 82 and/or the plurality of longitudinal slots (refs. 82A, 82B, 82C, 82D—e.g., FIG. 7) at the outer perimeter of the actuation mechanism 80 may be greater than 115% of the outer diameter of the friction element 90 and/or the elastomeric rod disposed within the longitudinal slot 82 and/or the plurality of friction elements (refs. 90A, 90B, 90C, 90D—e.g., FIG. 7) and/or the elastomeric rods disposed within the plurality of longitudinal slots (refs. 82A, 82B, 82C, 82D—e.g., FIG. 7). In some embodiments, the circumferential length 89 of the longitudinal slot 82 and/or the plurality of longitudinal slots (refs. 82A, 82B, 82C, 82D e.g., FIG. 7) at the outer perimeter of the actuation mechanism 80 may be greater than 120% of the outer diameter of the friction element 90 and/or the elastomeric rod disposed within the longitudinal slot 82 and/or the plurality of friction elements (refs. 90A, 90B, 90C, 90D—e.g., FIG. 7) and/or the elastomeric rods disposed within the plurality of longitudinal slots (refs. 82A, 82B, 82C, 82D—e.g., FIG. 7). Other configurations are also contemplated.

In some embodiments, the circumferential length 89 of the longitudinal slot 82 and/or the plurality of longitudinal slots (refs. 82A, 82B, 82C, 82D—e.g., FIG. 7) at the outer perimeter of the actuation mechanism 80 may be less than 150% of the outer diameter of the friction element 90 and/or the elastomeric rod disposed within the longitudinal slot 82 and/or the plurality of friction elements (refs. 90A, 90B, 90C, 90D—e.g., FIG. 7) and/or the elastomeric rods disposed within the plurality of longitudinal slots (refs. 82A, 82B, 82C, 82D—e.g., FIG. 7). In some embodiments, the circumferential length 89 of the longitudinal slot 82 and/or the plurality of longitudinal slots (refs. 82A, 82B, 82C, 82D—e.g., FIG. 7) at the outer perimeter of the actuation mechanism 80 may be less than 145% of the outer diameter of the friction element 90 and/or the elastomeric rod disposed within the longitudinal slot 82 and/or the plurality of friction elements (refs. 90A, 90B, 90C, 90D—e.g., FIG. 7) and/or the elastomeric rods disposed within the plurality of longitudinal slots (refs. 82A, 82B, 82C, 82D—e.g., FIG. 7). In some embodiments, the circumferential length 89 of the longitudinal slot 82 and/or the plurality of longitudinal slots (refs. 82A, 82B, 82C, 82D—e.g., FIG. 7) at the outer perimeter of the actuation mechanism 80 may be less than 140% of the outer diameter of the friction element 90 and/or the elastomeric rod disposed within the longitudinal slot 82 and/or the plurality of friction elements (refs. 90A, 90B, 90C, 90D—e.g., FIG. 7) and/or the elastomeric rods disposed within the plurality of longitudinal slots (refs. 82A, 82B, 82C, 82D—e.g., FIG. 7). In some embodiments, the circumferential length 89 of the longitudinal slot 82 and/or the plurality of longitudinal slots (refs. 82A, 82B, 82C, 82D—e.g., FIG. 7) at the outer perimeter of the actuation mechanism 80 may be less than 135% of the outer diameter of the friction element 90 and/or the elastomeric rod disposed within the longitudinal slot 82 and/or the plurality of friction elements (refs. 90A, 90B, 90C, 90D—e.g., FIG. 7) and/or the elastomeric rods disposed within the plurality of longitudinal slots (refs. 82A, 82B, 82C, 82D—e.g., FIG. 7). Other configurations are also contemplated.

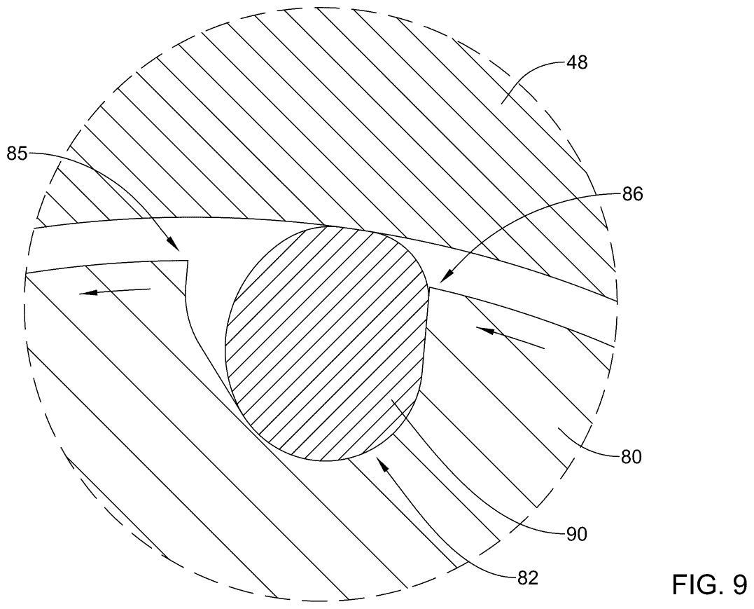

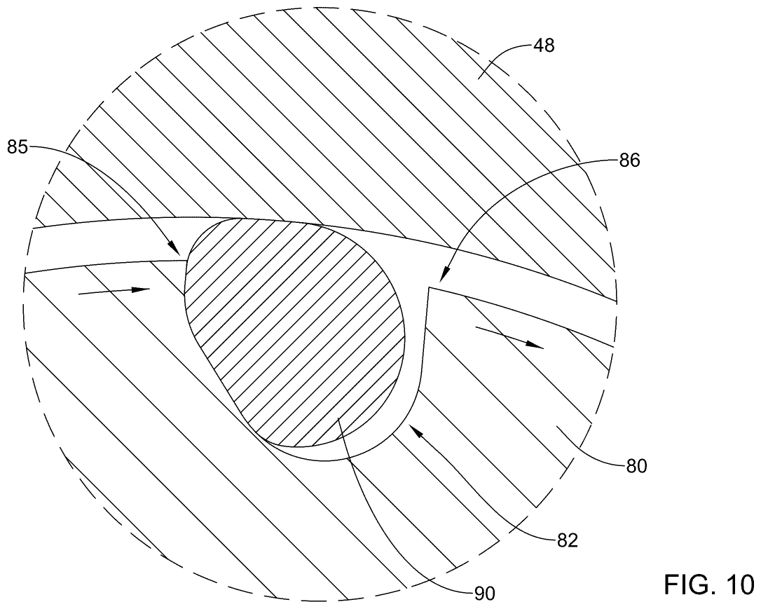

FIGS. 9-10 are partial cross-sectional views illustrating engagement of the friction element 90 and/or the elastomeric rod with the actuation mechanism 80 and the distal shell portion 48 as the actuation mechanism 80 is rotated relative to the distal shell portion 48, as evidenced by the arrows shown in the figures. It shall be noted that the first rotational direction (e.g., clockwise) and the second rotational direction (e.g., counterclockwise), as described herein, are opposite the rotational directions shown in FIGS. 9-10 because FIGS. 9-10 are shown as viewed in a distal to proximal direction, while the first rotational direction and the second rotational direction are defined above as viewed in a proximal to distal direction. Additionally, for context, in at least some embodiments, the distal shell portion 48 may be held substantially stationary while the actuation mechanism 80 may be rotated within and/or relative to the distal shell portion 48. Other configurations are also contemplated. As may be seen in FIGS. 9-10, the friction element 90 and/or the elastomeric rod may undergo some elastic deformation as a result of friction and/or compression as the actuation mechanism 80 is rotated relative to the distal shell portion 48.

In some embodiments, the friction element 90 and/or the elastomeric rod may be engaged with the actuation mechanism 80 and the distal shell portion 48. In some embodiments, the friction element 90 and/or the elastomeric rod may be engaged with the longitudinal slot 82 of the actuation mechanism 80 and the distal shell portion 48. In some embodiments, the friction element 90 and/or the elastomeric rod may cause and/or may be configured to cause a first amount of friction between the actuation mechanism 80 and the distal shell portion 48 when the actuation mechanism 80 is rotated relative to the distal shell portion 48 in the first rotational direction (e.g., clockwise—shown as counterclockwise in FIG. 9). In some embodiments, the friction element 90 and/or the elastomeric rod may cause and/or may be configured to cause a second amount of friction greater than the first amount of friction between the actuation mechanism 80 and the distal shell portion 48 when the actuation mechanism 80 is rotated relative to the distal shell portion 48 in the second rotational direction (e.g., counterclockwise—shown as clockwise in FIG. 10). Other configurations are also contemplated.

In some embodiments, the plurality of friction elements (refs. 90A, 90B, 90C, 90D—e.g., FIG. 7) and/or the elastomeric rods may be engaged with the actuation mechanism 80 and the distal shell portion 48. In some embodiments, the plurality of friction elements (refs. 90A, 90B, 90C, 90D—e.g., FIG. 7) and/or the elastomeric rods may be engaged with the plurality of longitudinal slots (refs. 82A, 82B, 82C, 82D—e.g., FIG. 7) of the actuation mechanism 80 and the distal shell portion 48. In some embodiments, the plurality of friction elements (refs. 90A, 90B, 90C, 90D—e.g., FIG. 7) and/or the elastomeric rods may cause and/or may be configured to cause a first amount of friction between the actuation mechanism 80 and the distal shell portion 48 when the actuation mechanism 80 is rotated relative to the distal shell portion 48 in the first rotational direction (e.g., clockwise—shown as counterclockwise in FIG. 9). In some embodiments, the plurality of friction elements (refs. 90A, 90B, 90C, 90D—e.g., FIG. 7) and/or the elastomeric rods may cause and/or may be configured to cause a second amount of friction greater than the first amount of friction between the actuation mechanism 80 and the distal shell portion 48 when the actuation mechanism 80 is rotated relative to the distal shell portion 48 in the second rotational direction (e.g., counterclockwise—shown as clockwise in FIG. 10). Other configurations are also contemplated.

In some embodiments, the friction element 90 and/or the elastomeric rod may be engaged in compression between the actuation mechanism 80 and the distal shell portion 48. In some embodiments, the friction element 90 and/or the elastomeric rod may be engaged in compression between the longitudinal slot 82 of the actuation mechanism 80 and the distal shell portion 48. In some embodiments, the plurality of friction elements (refs. 90A, 90B, 90C, 90D—e.g., FIG. 7) and/or the elastomeric rods may be engaged in compression between the actuation mechanism 80 and the distal shell portion 48. In some embodiments, the plurality of friction elements (refs. 90A, 90B, 90C, 90D—e.g., FIG. 7) and/or the elastomeric rods may be engaged in compression between the plurality of longitudinal slots (refs. 82A, 82B, 82C, 82D—e.g., FIG. 7) of the actuation mechanism 80 and the distal shell portion 48.

In some embodiments, the friction element 90 and/or the elastomeric rod may be engaged between the actuation mechanism 80 and the distal shell portion 48 in a first amount of compression when the actuation mechanism 80 is rotated relative to the distal shell portion 48 in the first rotational direction (e.g., clockwise—shown as counterclockwise in FIG. 9). In some embodiments, the friction element 90 and/or the elastomeric rod may be engaged between the actuation mechanism 80 and the distal shell portion 48 in a second amount of compression greater than the first amount of compression when the actuation mechanism 80 is rotated relative to the distal shell portion 48 in the second rotational direction (e.g., counterclockwise - shown as clockwise in FIG. 10). In some embodiments, the plurality of friction elements (refs. 90A, 90B, 90C, 90D—e.g., FIG. 7) and/or the elastomeric rods may be engaged between the actuation mechanism 80 and the distal shell portion 48 in the first amount of compression when the actuation mechanism 80 is rotated relative to the distal shell portion 48 in the first rotational direction (e.g., clockwise—shown as counterclockwise in FIG. 9). In some embodiments, the plurality of friction elements (refs. 90A, 90B, 90C, 90D—e.g., FIG. 7) and/or the elastomeric rods may be engaged between the plurality of longitudinal slots (refs. 82A, 82B, 82C, 82D—e.g., FIG. 7) of the actuation mechanism 80 and the distal shell portion 48 in the second amount of compression greater than the first amount of compression when the actuation mechanism 80 is rotated relative to the distal shell portion 48 in the second rotational direction (e.g., counterclockwise—shown as clockwise in FIG. 10).

In some embodiments, the first amount of compression may be at least 1%, at least 2%, at least 3%, at least 4%, etc. In some embodiments, the first amount of compression may be up to about 5%. In some embodiments, the second amount of compression may be at least 10%, at least 15%, at least 20%, at least 25%, etc. In some embodiments, the second amount of compression may be up to about 30%. Other configurations are also contemplated.

In some embodiments, a first amount of torque applied to the first rotatable knob 43 may be required to rotate the actuation mechanism 80 relative to the distal shell portion 48 in the first rotational direction (e.g., clockwise—shown as counterclockwise in FIG. 9). In some embodiments, a second amount of torque greater than the first amount of torque applied to the first rotatable knob 43 may be required to rotate the actuation mechanism 80 relative to the distal shell portion 48 in the second rotational direction (e.g., counterclockwise—shown as clockwise in FIG. 10).

In some embodiments, the first amount of torque may be 10 ounce-force inches (ozf-in) (0.0706 Newton meters (N-m)) of force or less. In some embodiments, the first amount of torque may be 8 ozf-in (0.0565 N-m) of force or less. In some embodiments, the first amount of torque may be 6 ozf-in (0.0424 N-m) of force or less. In some embodiments, the first amount of torque may be 5 ozf-in (0.0353 N-m) of force or less. In some embodiments, the first amount of torque may be 4 ozf-in (0.0282 N-m) of force or less. In some embodiments, the first amount of torque may be 3 ozf-in (0.0212 N-m) of force or less. In some embodiments, the first amount of torque may be 2 ozf-in (0.0141 N-m) of force or less. In some embodiments, the first amount of torque may be 1 ozf-in (0.0071 N-m) of force or less. Other configurations are also contemplated.

In some embodiments, the second amount of torque may be 18 ounce-force inches (ozf-in) (0.1271 N-m) of force or more. In some embodiments, the second amount of torque may be 20 ozf-in (0.1412 N-m) of force or more. In some embodiments, the second amount of torque may be 22 ozf-in (0.1553 N-m) of force or more. In some embodiments, the second amount of torque may be 24 ozf-in (0.1694 N-m) of force or more. In some embodiments, the second amount of torque may be 26 ozf-in (0.1836 N-m) of force or more. In some embodiments, the second amount of torque may be 28 ozf-in (0.1977 N-m) of force or more. In some embodiments, the second amount of torque may be 30 ozf-in (0.2118 N-m) of force or more. In some embodiments, the second amount of torque may be 32 ozf-in (0.2259 N-m) of force or more. In some embodiments, the second amount of torque may be 34 ozf-in (0.2400 N-m) of force or more. Other configurations are also contemplated.

In some embodiments, the second amount of torque may be at least 100% greater than the first amount of torque. In some embodiments, the second amount of torque may be at least 125% greater than the first amount of torque. In some embodiments, the second amount of torque may be at least 150% greater than the first amount of torque. In some embodiments, the second amount of torque may be at least 175% greater than the first amount of torque. In some embodiments, the second amount of torque may be at least 200% greater than the first amount of torque. In some embodiments, the second amount of torque may be at least 225% greater than the first amount of torque. In some embodiments, the second amount of torque may be at least 250% greater than the first amount of torque. In some embodiments, the second amount of torque may be at least 275% greater than the first amount of torque. In some embodiments, the second amount of torque may be at least 300% greater than the first amount of torque. In some embodiments, the second amount of torque may be at least 325% greater than the first amount of torque. In some embodiments, the second amount of torque may be at least 350% greater than the first amount of torque. In some embodiments, the second amount of torque may be at least 375% greater than the first amount of torque. In some embodiments, the second amount of torque may be at least 400% greater than the first amount of torque. Other configurations are also contemplated.

As may be seen in FIGS. 9-10, as the actuation mechanism 80 is rotated relative to the distal shell portion 48, the friction element 90 and/or the elastomeric rod may shift, move, roll, etc. toward and/or may engage with the first end 85 of the longitudinal slot 82 or the second end 86 of the longitudinal slot 82. The degree of compression on the friction element 90 and/or the elastomeric rod, and therefore the friction generated and/or the torque required to rotate the actuation mechanism 80 relative to the distal shell portion 48, may depend on which end of the longitudinal slot 82 the friction element 90 and/or the elastomeric rod is shifted, moved, rolled, etc. toward and/or is engaged with. Similarly, the amount of elastic deformation of the friction element 90 and/or the elastomeric rod may depend on which end of the longitudinal slot 82 the friction element 90 and/or the elastomeric rod is shifted, moved, rolled, etc. toward and/or is engaged with. In some embodiments, the friction element 90 and/or the elastomeric rod may undergo greater elastic deformation when shifted, moved, rolled, etc. toward and/or engaged with the first end 85 of the longitudinal slot 82 than when shifted, moved, rolled, etc. toward and/or engaged with the second end 86 of the longitudinal slot 82. In some embodiments, the friction element 90 and/or the elastomeric rod may undergo greater elastic deformation when the actuation mechanism 80 is rotated relative to the distal shell portion 48 in the first rotational direction (e.g., clockwise - shown as counterclockwise in FIG. 9) than when the actuation mechanism 80 is rotated relative to the distal shell portion 48 in the second rotational direction (e.g., counterclockwise - shown as clockwise in FIG. 10).

In some embodiments, more force may be required to shift the implant holding portion 60 from the closed configuration (e.g., FIGS. 2, 4) toward and/or to the open configuration (e.g., FIGS. 3, 5) than from the open configuration (e.g., FIGS. 3, 5) toward and/or to the closed configuration (e.g., FIGS. 2, 4). As such, loading the replacement heart valve implant 10 into the implant holding portion 60 may be easier than deploying the replacement heart valve implant 10 from the implant holding portion 60.

In use, the delivery device 30 may be advanced percutaneously through the vasculature to a position adjacent to the treatment site (e.g., the native heart valve, the aortic valve, etc.). For example, the delivery device 30 may be advanced through the vasculature and across the aortic arch to a position adjacent to the aortic valve. Alternative approaches to treat a defective aortic valve and/or other heart valve(s) are also contemplated with the delivery device 30. Orientation of the replacement heart valve implant 10, the delivery device 30, and/or elements thereof may be gleaned from FIGS. 1-3.

After positioning the replacement heart valve implant 10 and/or the expandable framework 12 at the treatment site and/or within the native heart valve (e.g., the aortic valve), the first rotatable knob 43 may be rotated relative to the handle assembly 40 and/or the distal shell portion 48 to translate the outer tubular member 52 and/or the proximal sheath 62 (e.g., the first portion of the elongate shaft assembly 50) proximally relative to the distal sheath 64 and/or the intermediate tubular member 56, thereby exposing and/or deploying the proximal portion of the replacement heart valve implant 10 from the implant holding portion 60 of the delivery device 30. Rotation of the first rotatable knob 43 in the second rotational direction may be configured to deploy the proximal portion of the replacement heart valve implant 10 from the implant holding portion 60 of the delivery device 30. In some embodiments, rotation of the first rotatable knob 43 in the second rotational direction to deploy the proximal portion of the replacement heart valve implant 10 from the implant holding portion 60 of the delivery device 30 may involve a series of slow, relatively short turns adding up to about 540 degrees of rotation, about 720 degrees of rotation, about 900 degrees of rotation, etc., using a pinch grip. Other configurations are also contemplated.

After exposing and/or deploying the proximal portion of the replacement heart valve implant 10 from the implant holding portion 60 of the delivery device 30, positioning of the replacement heart valve implant 10 and/or the expandable framework 12 relative to the treatment site (e.g., the native heart valve, the aortic valve, etc.) may be verified using appropriate visualization means. In some embodiments, the replacement heart valve implant 10 may be recaptured and/or repositioned at and/or within the treatment site (e.g., the native heart valve, the aortic valve, etc.) as necessary.

After verifying the positioning of the replacement heart valve implant 10 and/or the expandable framework 12 relative to the treatment site (e.g., the native heart valve, the aortic valve, etc.), and/or concluding that moving forward with the implantation procedure is desired, the second rotatable knob 44 may be rotated in the second rotational direction to translate the inner shaft 54 and/or the distal sheath 64 (e.g., the second portion of the elongate shaft assembly 50) distally relative to the proximal sheath 62 and/or the intermediate tubular member 56, thereby exposing and/or deploying the distal portion of the replacement heart valve implant 10 from the implant holding portion 60 of the delivery device 30. Rotation of the second rotatable knob 44 in the second rotational direction may be configured to deploy the distal portion of the replacement heart valve implant 10 from the implant holding portion 60 of the delivery device 30. In some embodiments, rotation of the second rotatable knob 44 in the second rotational direction to deploy the distal portion of the replacement heart valve implant 10 from the implant holding portion 60 of the delivery device 30 may involve a fast, relatively large degree of rotation using a power grip. In some embodiments, the relatively large degree of rotation may be between about 90 degrees and about 120 degrees, between about 95 degrees and about 115 degrees, between about 100 degrees and about 110 degrees, etc. In one non-limiting example, the relatively large degree of rotation may be about 103 degrees. Other configurations are also contemplated.

The materials that can be used for the various components of the implant delivery device and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the delivery device, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A delivery device for delivering a replacement heart valve implant to a native heart valve, comprising:
a handle assembly and an elongate shaft assembly extending distally from the handle assembly, wherein the handle assembly comprises:
a first rotatable knob configured to rotate around a central longitudinal axis of the handle assembly;
an actuation mechanism operatively engaged with the first rotatable knob;
a distal shell portion disposed radially outward of the actuation mechanism; and
a friction element engaged with the actuation mechanism and the distal shell portion;
wherein the first rotatable knob is configured to rotate the actuation mechanism relative to the distal shell portion to axially translate a first portion of the elongate shaft assembly along the central longitudinal axis;
wherein a first amount of torque applied to the first rotatable knob is required to rotate the actuation mechanism relative to the distal shell portion in a first rotational direction, and a second amount of torque applied to the first rotatable knob greater than the first amount of torque applied to the first rotatable knob is required to rotate the actuation mechanism relative to the distal shell portion in a second rotational direction.

2. The delivery device of claim 1, wherein the actuation mechanism comprises a longitudinal slot formed in an outer surface of the actuation mechanism proximate a distal end of the actuation mechanism.

3. The delivery device of claim 2, wherein a radial depth of the longitudinal slot is tapered in a circumferential direction.

4. The delivery device of claim 18, wherein the radial depth of the longitudinal slot is greater at a second end of the longitudinal slot than at a first end of the longitudinal slot.

5. The delivery device of claim 2, wherein the friction element is disposed within the longitudinal slot.

6. The delivery device of claim 5, wherein the friction element extends radially outward of the outer surface of the actuation mechanism.

7. The delivery device of claim 1, wherein the friction element comprises an elastomeric rod.

8. The delivery device of claim 1, wherein the friction element has a circular cross-section.

9. The delivery device of claim 1, wherein rotation of the actuation mechanism in the first rotational direction is configured to axially translate the first portion of the elongate shaft assembly distally relative to the handle assembly, and rotation of the actuation mechanism in the second rotational direction is configured to axially translate the first portion of the elongate shaft assembly proximally relative to the handle assembly.

10. The delivery device of claim 1, wherein the handle assembly comprises a second rotatable knob configured to rotate around the central longitudinal axis of the handle assembly to axially translate a second portion of the elongate shaft assembly along the central longitudinal axis.

11. The delivery device of claim 10, wherein rotation of the second rotatable knob in the first rotational direction is configured to axially translate the second portion of the elongate shaft assembly proximally relative to the handle assembly, and rotation of the second rotatable knob in the second rotational direction is configured to axially translate the second portion of the elongate shaft assembly distally relative to the handle assembly.

12. A delivery device for delivering a replacement heart valve implant to a native heart valve, comprising:
a handle assembly and an elongate shaft assembly extending distally from the handle assembly, wherein the handle assembly comprises:
a first rotatable knob configured to rotate around a central longitudinal axis of the handle assembly;
an actuation mechanism operatively engaged with the first rotatable knob;
a distal shell portion disposed radially outward of the actuation mechanism; and
a plurality of friction elements engaged with the actuation mechanism and the distal shell portion;
wherein the first rotatable knob is configured to rotate the actuation mechanism relative to the distal shell portion to axially translate a portion of the elongate shaft assembly along the central longitudinal axis;
wherein a first amount of torque applied to the first rotatable knob is required to rotate the actuation mechanism relative to the distal shell portion in a first rotational direction, and a second amount of torque applied to the first rotatable knob greater than the first amount of torque applied to the first rotatable knob is required to rotate the actuation mechanism relative to the distal shell portion in a second rotational direction.

13. The delivery device of claim 12, wherein the actuation mechanism comprises a plurality of longitudinal slots formed in an outer surface of the actuation mechanism proximate a distal end of the actuation mechanism.

14. The delivery device of claim 13, wherein each friction element of the plurality of friction elements is disposed within a separate longitudinal slot of the plurality of longitudinal slots.

15. A delivery device for delivering a replacement heart valve implant to a native heart valve, comprising:
a handle assembly and an elongate shaft assembly extending distally from the handle assembly, wherein the handle assembly comprises:
a first rotatable knob configured to rotate around a central longitudinal axis of the handle assembly;
an actuation mechanism operatively engaged with the first rotatable knob;
a distal shell portion disposed radially outward of the actuation mechanism; and
a friction element disposed within a longitudinal slot formed in an outer surface of the actuation mechanism, wherein the friction element is engaged in compression between the actuation mechanism and the distal shell portion;
wherein the first rotatable knob is configured to rotate the actuation mechanism relative to the distal shell portion to axially translate a first portion of the elongate shaft assembly along the central longitudinal axis;
wherein the friction element is engaged between the actuation mechanism and the distal shell portion in a first amount of compression when the actuation mechanism is rotated relative to the distal shell portion in a first rotational direction, and the friction element is engaged between the actuation mechanism and the distal shell portion in a second amount of compression greater than the first amount of compression when the actuation mechanism is rotated relative to the distal shell portion in a second rotational direction.

16. The delivery device of claim 15, wherein the first amount of compression is at least 1% and the second amount of compression is at least 10%.

17. The delivery device of claim 15, wherein a first amount of torque applied to the first rotatable knob is required to rotate the actuation mechanism relative to the distal shell portion in the first rotational direction, and a second amount of torque applied to the first rotatable knob greater than the first amount of torque applied to the first rotatable knob is required to rotate the actuation mechanism relative to the distal shell portion in the second rotational direction.

18. The delivery device of claim 17, wherein the second amount of torque is at least 200% greater than the first amount of torque.

19. The delivery device of claim 15, wherein the friction element comprises an elastomeric rod having a circular cross-section defining an outer diameter of the elastomeric rod.

20. The delivery device of claim 19, wherein the longitudinal slot comprises a first circumferential wall and a second circumferential wall collectively defining a circumferential length of the longitudinal slot at an outer perimeter of the actuation mechanism;
wherein the circumferential length of the longitudinal slot at the outer perimeter of the actuation mechanism is greater than 110% of the outer diameter of the elastomeric rod and less than 140% of the outer diameter of the elastomeric rod.
